(19) United States
(12) Patent Application Publication
(10) Pub. No.: US 2024/0252565 A1
(43) Pub. Date: Aug. 1, 2024

(54) A WATER SOLUBLE OR WATER DISPERSIBLE BOLUS ARTICLE

(71) Applicant: BIMEDA ANIMAL HEALTH LIMITED, Carrickmines, Dublin (IE)

(72) Inventors: Daniel Peter TIERNEY, Blackrock (IE); Donal Thomas Martin TIERNEY, Blessington (IE)

(73) Assignee: BIMEDA ANIMAL HEALTH LIMITED, Carrickmines, Dublin (IE)

(21) Appl. No.: 18/560,451

(22) PCT Filed: May 27, 2021
(86) PCT No.: PCT/EP2021/064292

§ 371 Date: Jan. 3, 2024

(30) Foreign Application Priority Data
May 14, 2021 (GB) 2106921.6
May 14, 2021 (GB) 2106923.2

(51) Int. Cl. A61K 36/02; A23K 40/20; A61K 9/00; A61K 9/19; A61K 31/02; A61K 31/336; A61K 47/12

(52) U.S. Cl. CPC A61K 36/02; A23K 40/20; A61K 9/0068; A61K 9/19; A61K 31/02; A61K 31/336; A61K 47/12

(57) ABSTRACT

A water soluble or water dispersible rumen bolus article suitable for oral administration to a reticulo rumen of a ruminant animal is described. The rumen bolus article comprises a water soluble or water dispersible matrix and seaweed or a seaweed extract enriched in at least one bioactive agent, wherein the rumen bolus article when administered to the reticulo rumen of the ruminant animal is configured to release the seaweed or seaweed extract over a sustained period of time. The seaweed extract is enriched in one or more bioactive agents selected from a vitamin, a mineral, an essential amino acid, an antioxidants, fibre, and a lipid. Bioactive agent generally constitutes at least 5 or 10% of the seaweed extract by weight. The seaweed extract is a lyophilised seaweed extract.

### FIELD OF THE INVENTION

The present invention relates to a water-soluble or water dispersible bolus article, and a method of manufacturing a water soluble/dispersible bolus article.

### SUMMARY OF THE INVENTION

The invention relates to a bolus article, especially a rumen bolus article, comprising a matrix that is generally water dispersible or water soluble and seaweed or a seaweed extract. The article is water soluble or water dispersible to release the seaweed or extract thereof over a sustained period in a stomach of an animal, especially a reticulo rumen of a ruminant animal. The seaweed may be processed in any way, for example dried or macerated.

In any embodiment, the rumen bolus comprises a seaweed extract that is typically enriched in a bioactive agent. The term bioactive agent means an agent that is present in seaweed or a seaweed extract and that is biologically or chemically active in an animal that receives the bolus article. Examples include nutrients and nutricines. Nutrients are the usually recognised components of feed such as carbohydrates, proteins, fats, minerals and vitamins, for example micronutrients such as a vitamin (A, B, C, E or K) and a mineral (iodine, manganese, iron, copper, zinc, sodium, calcium or magnesium), amino acids (including essentially amino acids such as tyrosine), lipids (including omega-3 fatty acids), protein and fibre (including sulphated polysaccharides). Nutricines are components of animal feeds which exert a beneficial effect upon health rather than contribute directly to nutrition. Important nutricines are; antioxidants (flavonoids, carotenoids, fucoxanthin), emulsifiers, enzymes, flavours, organic acids, prebiotics, probiotics.

In any embodiment, the seaweed extract is enriched in minerals.

In any embodiment, the seaweed extract is enriched in vitamins.

In any embodiment, the seaweed extract is enriched vitamins and minerals.

In any embodiment, the seaweed extract is enriched in thiamine.

In any embodiment, the seaweed extract is enriched in fucoxanthin.

In any embodiment, the seaweed extract is enriched in fucoidin.

In any embodiment, the seaweed extract is enriched in bromoform.

In any embodiment, the seaweed extract is enriched in fibre.

In any embodiment, the seaweed extract is enriched in omega-3 fatty acid.

In any embodiment, the seaweed extract is enriched in an organic acid.

In any embodiment, the seaweed extract is enriched in an emulsifier.

In any embodiment, the seaweed extract is enriched in an enzyme.

In any embodiment, the seaweed or extract thereof constitutes at least 10%, 20%, 30%, 40%, 50% or 60% of the bolus article by weight.

In any embodiment, the bioactive agent (or class of bioactive agents, e.g. vitamins or minerals) constitutes at least 10%, 20%, 30%, 40%, 50% or 60% of the seaweed extract by weight.

In any embodiment, the seaweed extract is lyophilised.

In any embodiment, the seaweed is a species of Fucus, Pelvetia, Ascophyllum, Asparagopsis, Laminaria, Saccharina, Sacchorhiza, Palmaria, Chondrus and Mastocarpus. The seaweed may be red, brown or green seaweed. Examples of red seaweed include Nori, Corallina, Palmaria palmata, Chondrus crispus and Mastocarpus stellatus. Kappaphycus, Betaphycus, Graciliania, Gelidum, and Pterocladia. Examples of brown seaweed include Ascophyllum nodosum and Laminaria hyperborea. Saccharina japonica, and Undaria pinnatifida. Examples of green seaweeds include Trebouxiophyceae (mostly subaerial and freshwater), Chlorophyceae (mostly freshwater), Bryopsidophyceae (seaweeds), Dasycladophyceae (seaweeds), Siphoncladophyceae (seaweeds), and Ulvophyceae (seaweeds).

In any embodiment, the bolus article comprises 1-50 g, 1-20 g, 5-15 g bioactive agent and is configured to release 10-200 mg 50-150 mg or 80-120 mg bioactive agent per day, especially in the reticulo rumen of a ruminant animal.

In any embodiment, the bolus article comprises a water soluble matrix in which the water soluble matrix comprises a water soluble glass.

In any embodiment, water soluble glass comprises a carboxylate glass.

In any embodiment, the carboxylate glass is formed from one or more metal carboxylates having a melting point of less than 300° C.

In any embodiment, water soluble glass comprises a polymer bonded glass.

The invention also provides a bolus article (especially a rumen bolus article) according to the invention, for use in a method of administering seaweed, a seaweed extract, or a bioactive agent contained in seaweed to an animal (especially a ruminant animal) by sustained release. The method generally comprises orally administering the bolus article to the stomach, or the reticulo rumen, of the animal wherein the seaweed, seaweed extract or bioactive agent is released from the bolus article in the animal over a sustained period of time.

Other aspects and preferred embodiments of the invention are defined and described in the other claims set out below.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

“Bolus article”: Bolus articles are described in the literature and are used to deliver active agents over a sustained period of time to animals. The articles are implanted into the gastrointestinal track of the animal, generally the stomach of monogastric animals, but more usually into the reticulo rumen of a ruminant animal such as a goat, sheep or cow (e.g. a rumen bolus). The bolus article generally is formed with a water soluble or water dispersible matrix or shell that breaks up over time in the stomach or reticulo rumen of an animal releasing an active agent contained within the matrix over a sustained period. They are often employed for sustained release of micronutrients and other active agents. Water soluble glasses, waxes and other materials are used as matrix materials. Rumen bolus articles are described in the following documents: GB2163346, EP0042219, CN104523681, GB2037735 (all water soluble glass bolus articles), U.S. Pat. No. 5,720,972 (wax coat), U.S. Pat. No. 5,322,692 (barium sulphate) and GB2333451 (wax coat). In one embodiment, the bolus article is a glass bolus article. In one embodiment, the bolus article is a non-glass bolus article.

“Water soluble or water dispersible” means that the bolus article is configured to break down during a sustained period of time in the digestive tract (e.g. the reticulo rumen) of the animal by dissolving (e.g. water soluble glass bolus) or dispersing to thereby release the seaweed or seaweed extract. The sustained period of time is generally at least one week and up to 15 weeks, for example 1-10, 1-5, 1-3, 2-10, 2-5 or 2-4 weeks.

As used herein, the term “water-soluble glass bolus article” refers to a solid article suitable for administration to the stomach or reticulo rumen of an animal that comprises water soluble glass, in one embodiment carboxylate glass (or another low-temperature glass forming material). Metal carboxylates such as acetates, propanoates and butanoates can be melted to a glass and have a lower melting temperature that phosphate-based glass materials, allowing thermally sensitive active agents such as polysaccharides to be formulated into a glass bolus. The metal carboxylate (or mixture of metal carboxylates) employed generally have a melting point of less than 300° C., 250° C. or 200° C. In one embodiment, the glass if formed without the bioactive agent, and then milled into particles and the bioactive agent and optionally a binder is added, and the mixture is formed into a suitable article (generally by pressing) and the article is then sintered to form the glass bolus article. In any embodiment, the sintered article comprises a binder. In another embodiment, the glass forming carboxylate(s) are mixed with the bioactive agent, melted to a liquid glass (typically at a temperature of less than 149° C.), and then cast into a glass bolus article. In any embodiment, the article is sintered.

In any embodiment, the bolus article has a weight of 10 to 200 g, 50 to 200 g, 10 to 50 g, 50 to 150 g, 75 to 125 g, and ideally about 90 to 110 g. In any embodiment, the bolus article contains 1-20%, 1-5%, 5-15%, 5-10%, 10-15%, 15-20% bioactive agent by weight. In any embodiment, the bolus article is configured to disintegrate in a stomach, for example a reticulo rumen, of the animal over a period of at least 1, 2, 3, 4, 5, 10, 15, 20, 25, 30, 35, 40, 45, 50, 55 or 60 days, for example 1 to 150 days, 10 to 120 days, 30 to 90 days, 40 to 80 days, 50 to 70 days, 1 to 5 days, 1 to 10 days, 10 to 20 days, 20 to 30 days, 30 to 40 days, 40 to 60 days, 50 to 70 days, or 50 to 60 days. In any embodiment, the bolus article is configured to release bioactive agent in a stomach, for example a reticulo rumen of the animal at a rate of to 500 mg, 10 to 200 mg, 50 to 150 mg, 80 to 120 mg, per day. In any embodiment, the bolus article has a specific gravity of 1 to 7, 1 to 6, 1 to 5, 1 to 4, 2 to 5, 2 to 4 g/cm3. In any embodiment, the bolus article has a surface area of 10 to 150, 10 to 100, 10 to 50, or 50 to 100 cm2. In any embodiment, the bolus article has a curved diameter of 10 to 50, 10 to 25, 25-50 mm.

As used herein, the term “carboxylate glass” refers to a glass formed when one or a mixture of metal carboxylates are heated to their melting temperature or above and allowed cool. In any embodiment, the carboxylate glass is selected from an acetate glass, a propanoate glass, a butanoate glass, and a mixed anion glass. The mixed anion glass may include carboxylates selected from an acetate, a propanoate, a butanoate and an octanoate. In any embodiment, the carboxylate glass comprises (or is formed from) a mixture of metal carboxylates, for example a mixture of metal acetates or a mixture of metal propanoates. Examples of carboxylates glasses are provided in Table 1 below. In any embodiment, the carboxylate glass comprises (or is formed from) at least 2, 3 or 4 metal carboxylates. In any embodiment, the cation of the metal carboxylate is selected from sodium, potassium, calcium and zinc, although other metals may be employed

TABLE 1

| | Liquidus | Estimated | |
|---|---|---|---|
| | temperature | duration of | Density |
| Glass composition ratio | (° C.) | glass | g/cm3 |
| Acetate glass | 140 | 2 hours | 1.48 |
| Sodium acetate | | | |
| Potassium acetate 1.1.1 | | | |
| Calcium acetate | | | |
| Propanoate glass | 180 | 8 hours | 1.32 |
| Sodium propanoate 1.1 | | | |
| Calcium propanoate | | | |
| Mixed anion glass | 160 | 2 weeks | 1.18 |
| Sodium butanoate | | | |
| Calcium propanoate 1.2.2 | | | |
| Sodium octanoate | | | |

In any embodiment, the seaweed extract is enriched in a natural bioactive agent. The term “natural bioactive agent” means an agent that is isolated from seaweed and is biologically or chemically active in an animal that receives the bolus article. Examples include nutrients and nutricines. Nutrients are the usually recognised components of feed such as carbohydrates, proteins, fats, minerals and vitamins, for example micronutrients such as a vitamin (A, B, C, E or K) and a mineral (iodine, manganese, iron, copper, zinc, sodium, calcium or magnesium), amino acids (including essentially amino acids such as tyrosine), lipids (including omega-3 fatty acids), protein and fibre (including sulphated polysaccharides). Nutricines are components of animal feeds which exert a beneficial effect upon health rather than contribute directly to nutrition. Important nutricines are; antioxidants (flavonoids, carotenoids, fucoxanthin), emulsifiers, enzymes, flavours, organic acids, prebiotics, probiotics. Bioactive agents found in seaweeds are described in Løvstad Holdt et al (Journal of Applied Physiology. 23, 543-597).

Methods of extracting saccharides (including sulphated polysaccharides) from seaweed are described in NL2009482, RU2197840, U.S. Pat. No. 8,007,838, KR20110133000, JP2003155244, CN103314014, KR20070020116, JP4571540, JP2010519383, GB727013, GB190525187, Antioxidants (Basel, Switzerland), Issue: 5, Volume: 8, Pages: 129. May 14, 2019,

Methods of extracting antioxidants from seaweed are described in CN104650191, IN294451, IN346531, JP2013203737, CN11267945, Annual review of food science and technology, Issue: 1, Volume: 10, Pages: 541-568. Jan. 23, 2019, Food Science and Technology Research, Issue: 2, Volume: 18, Pages: 251-257. 2012, Marine drugs, Issue: 5, Volume: 18, Pages: 250. May 11, 2020, Journal of agricultural and food chemistry, Issue: 16, Volume: 52, Pages: 4993-4997. Aug. 11, 2004, https://www.lens.org/lens/scholar/article/055-519-306-259-653/main, International journal of molecular sciences, Issue: 12, Volume: 17, Pages: 1988,

Methods of extracting minerals from seaweed are described in U.S. Pat. No. 1,051,984, KR20100025721, CN1095293, KR2007099792, KnE Life Sciences, Issue: 1, Volume: 2, Pages: 19. Feb. 1, 2015,

Methods of extracting lipids and/or fatty acids from seaweed are described in Food Chemistry Volume 65, Issue 3, May 1999, Pages 399-403, Supercritical Fluid Processing of Food and Biomaterials, Pages: 214-222. 1994, Journal of Wuhan Polytechnic University, 2000, Food Science and Technology International, 2000, OCL, Issue: 5, Volume: 25, Oct. 19, 2018,

Methods of extracting fibre from seaweed are described in JP2019135942, CN111019009, CN107522795, Trends in Food Science & Technology, Volume: 92, Pages: 46-64. 2019,

Methods of extracting amino acids from seaweed are described in CN107254496, CN105146470,

Methods of extracting bioactive agents from seaweed are described in EP1286680, CN106942739, CN104341536, KR100312547, US2007224215, KR0178449, JP11228602, KR1005769688, IN20171108874, Sustainable Seaweed Technologies, Pages: 315-370. 2020, Handbook of Marine Macroalgae, Pages: 221-228. Nov. 21, 2011, Journal of Food Bioactives, Volume: 2, Jun. 30, 2018, Algal Research, Volume: 48, Pages: 101909. 2020, Bioactive Compounds from Marine Foods, Pages: 269-312. Oct. 4, 2013, Environmental and Climate Technologies, Issue: 2, Volume: 24, Pages: 178-195. Sep. 1, 2020, Journal of Bioscience and Bioengineering, Volume: 108, 2009, Ultrasonics sonochemistry, Volume: 23, Pages: 308-316. Oct. 12, 2014, Journal of chromatography. B, Analytical technologies in the biomedical and life sciences, Volume: 1092, Pages: 152-157. Jun. 7, 2018, Methods in molecular biology (Clifton, N.J.), Volume: 1308, Pages: 145-150. 2015.

Methods of extracting polyphenols from seaweed are described in CN105362300, CN105193863, Journal of Food Processing and Preservation, Issue: 12, Volume: 43, Oct. 23, 2019, https://www.lens.org/lens/scholar/article/112-322-757-948-411/main,

As used herein the term “enriched in bioactive agent” as applied to a seaweed extract means that the concentration of the bioactive in the extract is significantly greater than the concentration of the bioactive in dried seaweed (dried to a water content (Aw) of 0.5), typically at least 2, 5, 10, 20, 30, 40, 50 or 100 times greater. In any embodiment, the bioactive agent constitutes at least 0.1%, 0.5%, 1%, 1.5%, 2%, 5%, 10%, 20%, 30%, 40% or 50% by weight of the seaweed extract. In any embodiment, bioactive agent constitutes at least 60%, 70% or 80% by weight of the seaweed extract. In any embodiment, the bromoform is a methanolic extract of seaweed.

The invention also provides a method of forming a water-soluble glass bolus article. In one embodiment, the method comprises melting a metal carboxylate to form a liquid carboxylate glass, cooling the liquid carboxylate to provide a solid carboxylate glass, milling the solid carboxylate glass to provide carboxylate glass particles, mixing the carboxylate glass particles with bioactive agent, and sintering the mixture to form a sintered water-soluble glass bolus article. In another embodiment, the glass forming carboxylate(s) are mixed with the bioactive agent, melted to a liquid glass (typically at a temperature of less than 149° C.), and then cast into a glass bolus article. In any embodiment, the melting step is performed at a temperature of 50 to 300° C., 100 to 300° C., 100 to 250° C., 100 to 200° C., 150 to 300° C., 200 to 300° C., 150 to 250° C. or 200 to 250° C. In any embodiment, the sintering step is performed at a temperature of 20 to 250° C., 50 to 250° C., 100 to 250° C., 150 to 250° C., 50 to 200° C., 100 to 200° C. or 150 to 200° C. In any embodiment, the sintering step comprises an annealing step. In any embodiment, the annealing step is performed at a temperature of 20 to 200° C., 50 to 200° C., 100 to 200° C., 150 to 200° C., 50 to 1500° C., or 150 to 200° C. In any embodiment, the milling step is configured to provide carboxylate glass particles having an average particle size of 0.1 to 1 mm, 0.5 to 1 mm, 0.1 to 0.5 mm. In any embodiment, the mixing step comprises adding a binder to the carboxylate glass particles and bioactive agent. In any embodiment, the mixture of carboxylate glass particles, bioactive agent and optionally binder is granulated prior to the sintering step. Sintering or frittage is the process of compacting and forming a solid mass of material by heat and/or pressure without melting it to the point of liquefaction. It is described in German et al. (Encyclopedia of Materials: Science and Technology, 2001 and Sintering: from Empirical Observations to Scientific Principles, 2014. In another embodiment, a polymer may be used in a non-sintered bolus. In this case, the glass is prepared either as a carboxylate or phosphate glass, then milled to a particular size and mixed with bioactive agent and optionally a densifier, added to a mould, then the polymer is added and the materials pressed to solidify the mixture.

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Example 1

A mixture of 1:1:1 sodium acetate, potassium acetate and calcium acetate is mixed together and heated to 140° C. until the mixture liquifies. The liquid glass is allowed to cool and is then milled and screened to an average particle size of 0.1 to 1 mm. Seaweed dried to a water activity (Aw) of 0.6 is prepared in a vacuum dryer. The Dried seaweed is added to the particulate glass in a weight ratio of 1 part seaweed extract to 6 parts particulate glass, mixed with any binding agent if applicable and pressed into a bolus of required dimensions. The pressed boluses are then sintered at a pre-determined temperature and annealed at a pre-determined temperature (obtained through analysis of the glass by differential scanning calorimetry).

### Example 2

A mixture of 1:1:1 sodium acetate, potassium acetate and calcium acetate is mixed together and heated to 140° C. until the mixture liquifies. The liquid glass is allowed to cool and is then milled and screened to an average particle size of 0.1 to 1 mm. Lyophilised seaweed extract is prepared according to the method of Example 1 of WO2020113279. The extract contains about 60% bromoform by weight. The lyophilized extract is added to the particulate glass in a weight ratio of 1 part seaweed extract to 6 parts particulate glass, mixed with any binding agent if applicable and pressed into a bolus of required dimensions. The pressed boluses are then sintered at a pre-determined temperature and annealed at a pre-determined temperature (obtained through analysis of the glass by differential scanning calorimetry).

### Example 2

The method of Example 1 is repeated except that the glass is formed from 1:1 mixture of sodium propanoate and calcium propanoate heated to 180ºC until the mixture liquifies.

### Example 3

The method of Example 1 is repeated except that the dried seaweed is replaced with a seaweed extract prepared according to a method of IN294451A1 that is rich in antioxidant and anti-inflammatory agents.

### Example 4

The method of Example 1 is repeated except that the dried seaweed is replaced with a seaweed extract prepared according to a method of GB355223 that is rich in minerals and alkaline alginates.

### Example 5

The method of Example 1 is repeated except that the dried seaweed is replaced with a seaweed extract prepared according to a method of CN107522795 that is rich in sulphonated polysaccharides.

### Example 6

The method of Example 1 is repeated except that the dried seaweed is replaced with a seaweed extract prepared according to a method of KR20110133000 that is enriched in fucoxanthin.

### Example 7

15 g of lyophilised seaweed extract is prepared according to the method of Example 1 of WO2020113279. The extract contains about 10 g bromoform. A 2:2:2:1 mixture of sodium acetate, potassium acetate, calcium acetate and lyophilised seaweed extract is mixed together and heated to 140° C. until the mixture liquifies. The liquid glass is cast into glass articles having a diameter of 1.5 cm, length of 4 cm, and weight of 27 g.

### Example 8

A carboxylate or phosphate glass is prepared and milled to a specific particle size. This glass is mixed with the bromoform and placed into a mould. The polymer binder is added to the mould and then pressed to solidify the bolus. (polymer bonded glass bolus).

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A water soluble or water dispersible rumen bolus article suitable for oral administration to a reticulo rumen of a ruminant animal comprising a water soluble or water dispersible matrix and seaweed or a seaweed extract enriched in at least one bioactive agent, wherein the rumen bolus article when administered to the reticulo rumen of the ruminant animal is configured to release the seaweed or seaweed extract over a sustained period of time.

2. A water soluble or water dispersible rumen bolus article according to claim 1, comprising a seaweed extract, in which the seaweed extract is enriched in a bioactive agent selected from: a vitamin; a mineral; an essential amino acid; an antioxidant; fibre; an organic acid; an enzyme; a prebiotic; a polysaccharide; and a lipid.

3. A water soluble or water dispersible rumen bolus article according to claim 1 or 2, in which the bioactive agent constitutes at least 5% of the seaweed extract by weight.

4. A water soluble or water dispersible rumen bolus article according to claim 1 or 2, in which the bioactive agent constitutes at least 10% of the seaweed extract by weight.

5. A water soluble or water dispersible rumen bolus article according to any preceding claim, in which the seaweed extract is lyophilised seaweed extract.

6. A water soluble or water dispersible rumen bolus article according to any preceding claim, in which the seaweed extract enriched in bromoform constitutes 1 to 20% by weight of the water soluble or water dispersible rumen bolus article.

7. A water soluble or water dispersible rumen bolus article according to any preceding claim, comprising 5-15 g bioactive agent and configured to release 10-200 mg bioactive per day in the reticulo rumen of a ruminant animal.

8. A water soluble or water dispersible rumen bolus article according to any preceding claim comprising a water soluble matrix and bioactive agent distributed throughout the matrix, in which the water soluble matrix comprises a water soluble glass.

9. A water soluble or water dispersible rumen bolus article according to claim 8 in which the water soluble glass comprises a carboxylate glass.

10. A water soluble or water dispersible rumen bolus article according to claim 8 in which the carboxylate glass is formed from one or more metal carboxylates having a melting point of less than 300° C.

11. A water soluble or water dispersible rumen bolus article according to claim 8 in which the water soluble glass comprises a polymer bonded phosphate glass.

12. A water soluble or water dispersible rumen bolus article according to any of claims 1 to 11, for use in a method of administering bioactive agent to a ruminant animal by sustained release, the method comprising orally administering the rumen bolus article to the reticulo rumen of the animal wherein the bioactive agent is released from the rumen bolus article in the reticulo rumen of the animal over a sustained period of time.

13. A water soluble or water dispersible rumen bolus article according to any of claims 1 to 11, for use of claim 12, in which the rumen bolus article comprises 5-15 g bioactive agent, and is configured to release 10-200 mg bioactive agent per day in the reticulo rumen of the animal for a sustained period of 20-100 days.

14. A water soluble or water dispersible rumen bolus article according to any of claims 1 to 11, for use of claim 12 or 13, in which the seaweed extract is enriched in a bioactive agent selected from: a vitamin; a mineral; an essential amino acid; an antioxidants; fibre; and a lipid.

15. A water soluble or water dispersible rumen bolus article according to any of claims 1 to 11, for use of claim 12 or 13, in which the seaweed extract is enriched in at least two bioactive agents selected from: a vitamin; a mineral; an essential amino acid; an antioxidants; fibre; and a lipid.

16. A water soluble or water dispersible rumen bolus article according to any of claims 1 to 11, for use of claim 12 or 13, in which the seaweed extract is enriched in at least three bioactive agents selected from: a vitamin; a mineral; an essential amino acid; an antioxidants; fibre; and a lipid.
